(19) United States
(12) Patent Application Publication
(10) Pub. No.: US 2022/0051766 A1
(43) Pub. Date: Feb. 17, 2022

(54) SYSTEMS AND METHODS FOR A MEMBER-CENTRIC HEALTH MANAGEMENT PLATFORM

(71) Applicant: Clear Spring Health Holdings, LLC, Park Ridge, IL (US)

(72) Inventor: Arthur Charles Carlos, III, Park Ridge, IL (US)

(21) Appl. No.: 17/399,737

(22) Filed: Aug. 11, 2021

Related U.S. Application Data
(60) Provisional application No. 63/064,018, filed on Aug. 11, 2020.

(51) Int. Cl. G16H 10/60; G16H 50/30

(52) U.S. Cl. CPC G16H 10/60; G16H 50/30

(57) ABSTRACT

An example method for selective content provision for members of a health management system involves receiving demographic information related to a member. The method also involves receiving health information related to the member. The health information includes a medical condition code, a health risk assessment, health insurance plan information, and health insurance claim data. The method also involves determining a member profile using the demographic information and the health information. The method also involves selecting content for the member based on the member profile. The method also involves providing the selected content to a computing device associated with the member.

### RELATED APPLICATION

This application claims priority from U.S. Provisional Application No. 63/064,018 filed on Aug. 11, 2020, the entirety of which is incorporated herein by reference.

### BACKGROUND

If one were compiling a list of least favorite activities, dealing with health insurance companies would be on that list, if not at the top of the list. Even today, health insurance companies are generally known for providing medical reimbursement, often through contentious interactions with some patients and/or medical providers. Most health insurance companies do not provide health services themselves or access to health services as an added benefit. The few companies that do provide health services, only provide services through interfaces that are difficult to navigate. While the intention of these few health insurance companies is good, the offered content is generic and not tailored to an individual patient or medical condition. As a result of these issues, online health services are typically underutilized by patients.

Additionally, there are hundreds of different telehealth providers that provide online health services. Most of these services are provided via a single tablet or smartphone-based application (e.g., an App) that focuses on one medical condition. For instance, there are a number of separate applications for helping patients manage diabetes, high blood pressure, obesity, depression, addiction, etc. However, each application is generally self-contained for a single medical condition and is not customized for any particular patient. This means that a patient with a certain condition has to locate the correct application in an application store, and then use that application exclusively for that condition. A patient with a rare medical condition may not be able to locate a corresponding application or may be unaware of the existence of such application. Further, as one can imagine, patients with application fatigue generally use such a health-based application less frequently over time, even applications prescribed or recommended by a healthcare provider. As a result, many telehealth applications are not as beneficial as intended.

### SUMMARY

Example system, methods, and apparatus are disclosed herein that use a member's health information as a basis for distributing patient-centric content.

In an example, a method for selective content provision for members of a health management system is provided. The method comprises receiving demographic information related to a member. The method also comprises receiving health information related to the member. The health information includes a medical condition code, a health risk assessment, health insurance plan information, and health insurance claim data. The method also comprises determining a member profile using the demographic information and the health information. The method also comprises selecting content for the member based on the member profile. The method also comprises providing the selected content to a computing device associated with the member.

In another example, a system is provided. The system includes a content database and a health management server. The health management server is configured to receive demographic information related to a member and to receive health information related to the member. The health information includes a medical condition code, a health risk assessment, health insurance plan information, and health insurance claim data. The health management server is also configured to determine a member profile using the demographic information and the health information. The health management server is also configured to select content from the content database for the member based on the member profile. The health management server is also configured to load the selected content onto a computing device designated for the member.

In another example, a non-transitory computer readable medium storing instructions is provided. The instructions, when executed on one or more processors, cause a computing system to receive demographic information related to a member and health information related to the member. The health information includes a medical condition code, a health risk assessment, health insurance plan information, and health insurance claim data. The instructions also cause the computing system to determine a member profile using the demographic information and the health information. The instructions also cause the computing system to select content for the member based on the member profile. The instructions also cause the computing system to provide, via a network, the selected content to a computing device associated with the member.

Additional features, advantages, and examples are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not necessarily include all of the advantages and/or aspects listed herein. Thus, the present disclosure expressly contemplates claiming individual and/or various combinations of the aspects and/or advantageous embodiments described herein. Moreover, it should be noted that the language used in the specification has been selected principally for readability and instructional purposes, and not to limit the scope of the inventive subject matter.

### BRIEF DESCRIPTION OF THE FIGURES

FIG. 1 is a diagram of a health management system, according to an example embodiment of the present disclosure.

FIG. 2 is a diagram of the health management system of FIG. 1 with un-provisioned personal computing devices, according to an example embodiment of the present disclosure.

FIG. 3 is a diagram of the health management system of FIG. 2 with at least two provisioned personal computing devices, according to an example embodiment of the present disclosure.

FIG. 4 is a diagram of a process performed by a health management server, according to an example embodiment of the present disclosure.

FIG. 5 is a flowchart of a process performed by a health management system, according to an example embodiment of the present disclosure.

FIG. 6 illustrates an example user interface provided to a member at a personal computing device associated with the member, according to an example embodiment of the present disclosure.

### DETAILED DESCRIPTION

Methods, systems, and apparatus are disclosed herein for a health management platform that enhances member engagement and provides health-related services that are specifically tailored for each individual member. Some example methods, systems, and apparatus provided herein may utilize a Wi-Fi enabled personal computing device or other type of computing device, such as a smartphone, tablet computer, laptop computer, desktop computer, among other examples, that is pre-loaded with software applications and/or other types of content primarily focused on enabling members to conduct telehealth visits, access educational content and basic benefit information, and to facilitate engagement with their medical plan. Additionally or alternatively, the computing device associated with the member may be configured remotely (e.g., via a network, to install or load the various applications, content, etc. on the computing device of the member. By supplying members with access to the disclosed health management platform via the personal computing device of the member, the methods, systems, and apparatus disclosed herein create a unique experience that increases member satisfaction while also improving health outcomes.

In an example, a method is provided that involves receiving demographic information and health information related to a member. As discussed herein, health information may include actuarial based underwriting data, such as a member's health data and corresponding underwriting classifications for certain identified medical conditions/diseases. The system, methods, and apparatus may also use predictive modeling using the health information to provide a health risk assessment and/or morbidity assessment for diseases/medical conditions that a member is likely to experience in the future or is currently experiencing. In some instances, the health information includes chronic condition codes, other medical codes (e.g., international classification of diseases (ICD) codes, etc.), member demographics, health risk assessment data, plan specific benefit design, and/or member claims data.

In some examples, the method involves using the health information and the demographic information to determine a member profile, which is assigned into one of a plurality of cohort groups. Based on a group assignment, one or more applications, personalized recommendations, and/or other content is loaded into a personal computing device associated with the member, such as a tablet computer or other type of computing device. The member uses the computing device as a portal or gateway for accessing the applications and other content that is specific to their condition, thereby providing more personalized telehealth service to individual members. In some examples, the method involves removing or adding certain applications and/or other content as a member's health changes over time, as detected by an insurance provider or other health management server. As discussed herein, since a member typically only has one insurance plan, but may have many different medical providers, example health management systems herein may be in an ideal position to aggregate a member's medical history/condition information from different past and current medical providers to formulate a comprehensive and dynamic member profile that accurately reflects the current (and/or predicted) condition of the member.

In some examples, the method involves providing connectivity between a member and a health insurance company through the computing device of the member. This can include providing a member portal with a dashboard with insurance plan information, such as payments being processed, deductible remaining, benefits remaining, etc. The portal may also include a dashboard with personal incentive and tracking information. Further, the computing device may include applications that enable a web chat, video chat, or phone call with a care management team, a telehealth provider, or a member's clinicians.

In some examples, the method involves computing a health index, such as a number between 0 and 100 for instance, that reflects the member's physical and mental health state based on the health information and the demographic information. For instance, the received health information may include health risk assessment (HRA) responses received from the member, scientific based questionnaires (e.g., SF-12 questionnaires, etc.), clinical events (e.g., primary care physician (PCP) visits, medical exams, vital measurements, prescriptions, vaccines, mind wellness scores, etc.), as well as other types of health information compiled by a system of the example method. The example method may then compute the health index as a weighted sum of values (e.g., values that represent each of the different health and/or demographic aspects of the member) that indicates an overall health state of the member. The example method may also involve using the health index to select content (e.g., applications, media, personalized recommendations, etc.) to add or remove at the computing device of the member. For instance, the health index (and/or other information such as risk cohort groups) can be used to select content from a database that maps the content to specific health conditions or health state of the member. As an example, a member that has a relatively high health index but an advanced age may be provided with applications or recommendations for scheduling a mind wellness checkup via telehealth or with a clinical team.

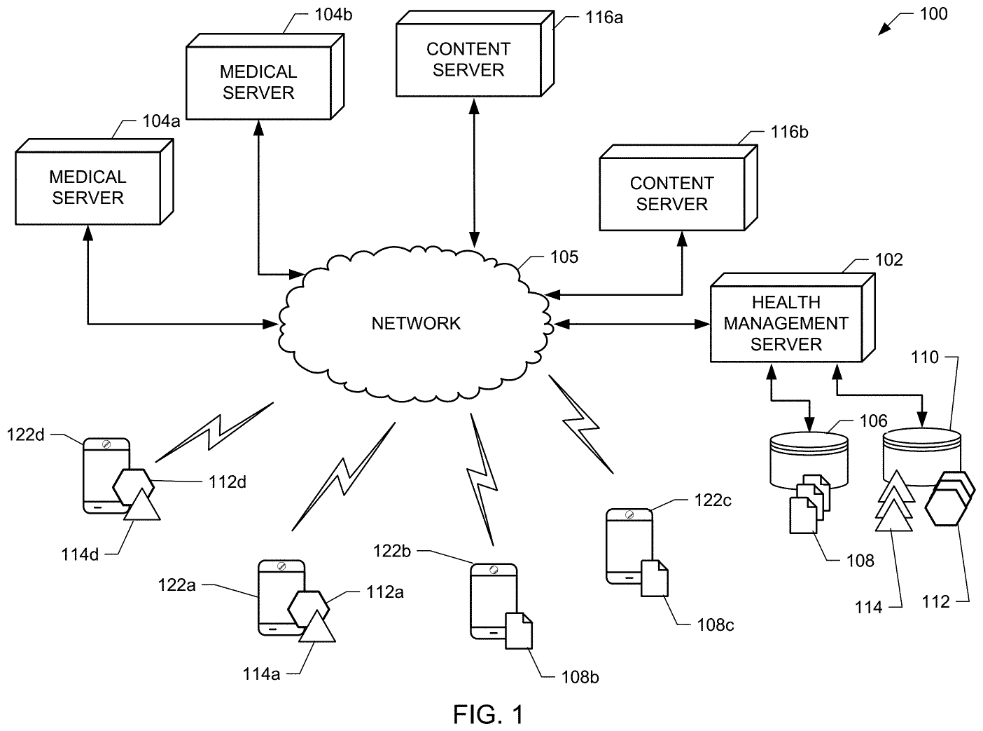

FIG. 1 shows a diagram of a health management system 100, according to an example embodiment of the present disclosure. In the illustrated example, the health management system 100 is implemented a computing system that includes a health management server 102 communicatively coupled (via a network 105) to one or more medical servers 104a, 104b, one or more content servers 116a, 116b, and one or more computing devices 122a, 122b, 122c, 122d. The system 100 also includes one or more memory devices 106, 110. As discussed herein, the health management server 102 receives health information (e.g., health insurance information) and demographic information (e.g., age, race, ethnicity, etc.) that is related to members for creating member profiles.

In some examples, the health management server 102 includes one or more machine learning algorithms or analytic processors for processing a member's health insurance information into a member profile. For example, the server 102 may include one or more processors, such as single core processors, multi-core processors, etc., and a memory (e.g., memory devices 106, 110, etc.) storing instructions that are executable by the one or more processors to perform the functions described in the present disclosure.

In the illustrated example, the health management server 102 may be specific for a single health insurance provider. In other examples, the system 100 can include other health management servers 102 for other health insurance providers. Further, the health management server 102 may provide services for more than one health insurance provider.

A member profile (e.g., 108, 108b, 108c) generated by the server 102 includes an indication of a member's health condition or health state. The member profile 108 may also include a predictive assessment of health conditions a member is likely to experience based on their current detected health status. In some instances, the member profile 108 may include a health risk assessment and/or morbidity assessment.

In an example, a member profile 108 may include a health index score computed for a member based on the health information and demographic information received by the server 102. For instance, a health index score for a member can be computed using the following equation:

Health Index=HRA responses*Historical claims*Demographics*Socioeconomic factors.

In this equation, each HRA response may be assigned a value. For instance, an HRA questionnaire may ask a user “How often do you feel depressed?” In response, the user selects one of the following options: “Not at all,” “Several days a month,” “more days than not,” “nearly every day.” Each option is associated with a value that characterizes the response (e.g., 100, 60, 50, 40). Thus, depending on the responses selected by the member in the HRA questionnaire, corresponding values can be inserted as the “HRA responses” variable(s) of the equation above. Similarly, historical claims (e.g., diabetes medication, demographics (e.g., age, gender, etc.), and socioeconomic factors (e.g., address, ethnicity, income level, etc.) can be obtained from the member (e.g., via a questionnaire), from health insurance information, or from other sources (e.g., medical servers 104, etc.), and can also be assigned a corresponding value (e.g., between 0 and 100, etc.) in the equation above. As such, the health index score can be computed for each member as a single value that represents an overall health state of the member.

In some examples, the system 100 and/or the server 102 can use the health index score to select content (e.g., health recommendations, applications, media, etc.) to add and/or remove from the respective computing device 122 of the member. For instance, in the illustrated example, device 122a is loaded with application 112a and content 114a that are selected for a first member associated with device 122a, and device 122d is loaded with application 122d and content 114d that are selected for a second member associated with device 122d.

In some examples, the system 100 and/or the server 102 may provide a user interface (e.g., web portal, etc.) at a particular computing device 122 that includes information from a particular member profile (e.g., 108b, 108c, etc.) of the member associated with the particular computing device (e.g., 122b, 122c, etc.).

As noted above, the example health management server 102 is communicatively coupled to one or more medical servers 104 for receiving at least some health information and demographic information for one or more members. The medical servers 104 may be associated with one or more medical systems, which transmit member claim data and/or other health information related to a member. The health information may be transmitted over the network 105, such as the Internet, a cellular network, other network, or a combination of one or more networks. The health management server 102 is configured to compile the received information with chronic or other medical condition codes (e.g., ICD codes), member demographics, health risk assessment data, and/or insurance plan information, which is stored in the memory device 106 as health insurance information or member profile 108. To that end, in some examples, the memory device 106 may be referred to herein as a member profile database 106.

The memory device 110 stores applications 112 and content 114. To that end, in some examples, the memory device 110 may be referred to herein as content database 108. The applications 112 are software programs that operate on a personal computing device 122 (when installed on the device 122). The content 114 includes documents, videos, audio, or other types of media content. In some examples, the content 114 also includes health recommendations associated with a specific risk cohort group. For example, a health recommendation 114 for members having an age greater than a threshold (e.g., 50 years, etc.) may include a reminder such as “schedule a mind wellness checkup via telehealth or with a clinical team.” Other examples are possible.

In some instances, the applications 112 and/or content 114 may be provided or otherwise made available through content servers 116. For example, the applications 112 may include fields or templates that access one or more application programming interfaces (APIs) at one or more content servers 116 for obtaining information to present at a computing device 122 of the member and/or for obtaining an application 112 to install at the computing device 122 of the member. Additionally or alternatively, the content 114 may include a hyperlink to content that is available from a host (not shown), such as a web server or other remote server.

In addition to applications for treating a medical condition, the applications 112 may also include software for interfacing with a health insurance company and/or one or more medical providers. The applications 112 can include, for example, applications for PCP contact information, care coordination contact information, broker contact information, case management contact information, member plan link, telemedicine provider, software game, hearing test, health club, weight management, over-the-counter (OTC) drug provider, health team, PCP visit scheduler, PCP search, dental provider search, and/or vision provider search. Further, each of the personal computing devices 122 may receive member materials (e.g., content 114) including, for example, an online/offline provider directory, provider mapquest, member portal, tracking for member incentivized programs that are tied to preventative care activity, company directory of service phone numbers/emails, evidence of coverage, summary of benefits, one touch-digital ID card, and/or customized chronic disease content, among other examples. Further, the applications 112 may include software for secure log-in, logo on boot up, web browser, link to appropriate plan landing page with secure log-in, web chat with a care management team, web chat with member services, appointment scheduling, Zoom®, and/or Web Ex®.

The memory devices 106 and 110 may include any computer-readable medium, such as random access memory (“RAM”), read only memory (“ROM”), flash memory, magnetic or optical disks, optical memory, or other storage media.

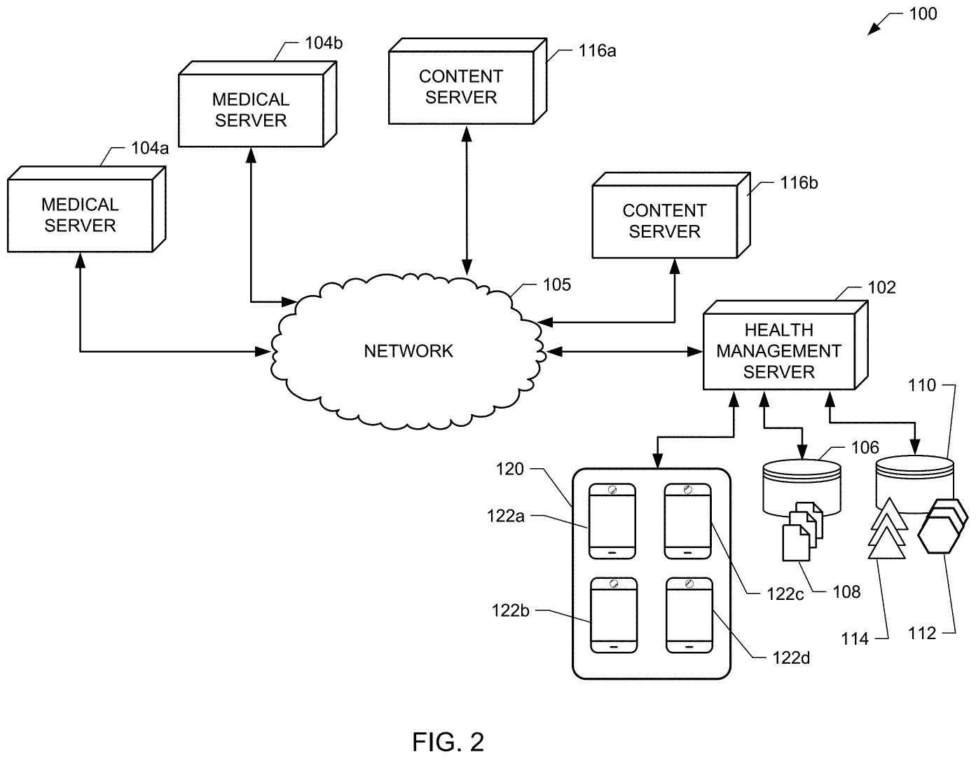

FIG. 2 illustrates an implementation of the example system 100 that includes a personal computing device repository 120, which includes personal computing devices 122. In particular, the implementation of the example system 100 illustrated in FIG. 2 enables pre-loading a member's computing device 122 with specific applications 112 and/or content 114 before providing the configured device 122 to the member. To that end, the repository 120 contains devices 122 that have not yet been provided to a member. The personal computing devices 122 may include any smartphone, tablet computer, laptop computer, desktop computer, smartwatch, etc.

When a personal computing device 122 is to be provisioned for a member, the example health management server 102 (e.g., by executing the algorithm 300 to operate the health management server), identifies which of the applications 112 and/or content 114 is to be installed onto a designated personal computing device (e.g., a personal computing device 122a) of a member. The server 102 then installs the applications 112 and/or content 114 onto the personal computing device 122a, 122b, 122c, and/or 122d designated for that particular member. Installation may also include registering the applications and/or content on behalf of the member using at least some of the health insurance information/member profile 108. The health management server 102 may also complete any software license registration requirements for the installed applications 112 and/or content 114. Further, the health management server 102 may perform an automated check to ensure the applications 112 and/or content 114 properly load.

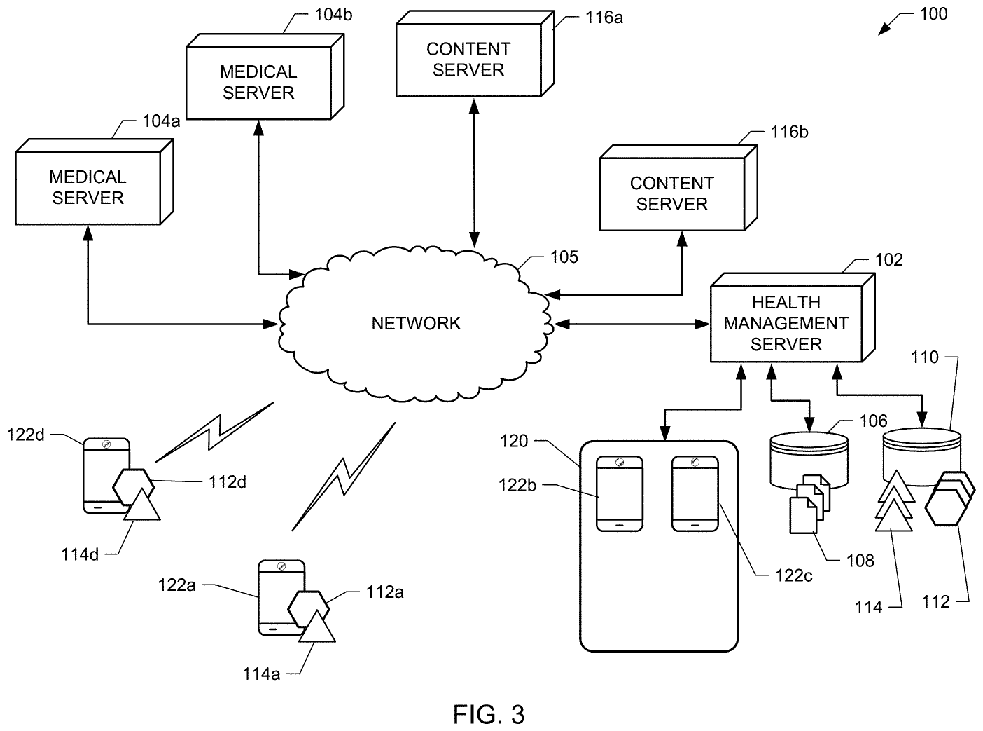

FIG. 3 shows an implementation of the health insurance system 100 where personal computing devices 122a and 122d have been provisioned and deployed for respective members, according to an example embodiment of the present disclosure. Each of the computing devices 122a and 122d has different configurations of applications 112 and content 114. For example, computing device 122a may have application 112a and content 114a loaded thereon based on application 112a and content 114a being mapped (e.g., in content database 110) to a risk cohort group that matches a member profile of the member associated with device 122a. Thus, the respective members are able to use the specific installed applications 112 and content 144 on their personal computing devices 122a and 122d to access their insurance information, access their health providers, and/or receive telemedicine treatments. In some examples, the personal computing devices 122a and 122d provide a portal or gateway to the health management server 102 and/or the content servers 116.

In some examples, a successful preventative care program is a critical element to managing a Medicare population or other health plan population. To that end, by engaging members in their care through the use of the personal computing device 122 for example, a health management team (via the server 102) will have an opportunity to create reliable touchpoints with members through a defined and robust communication system (e.g., the system 100). Health plans traditionally have challenges with telephonic outreach as up to date contact information is hard to maintain. More generally, example systems, methods, and apparatus of the present disclosure enable a higher success rate when outreaching to members by providing a dedicated member-centric communication interface (e.g., via the server 102 and the computing devices 122) between operators (e.g., health plan providers, etc.) of the system 100 and their members.

The proactive capabilities enabled by the system 100 include at least the following three categories. The first is a health risk assessment (“HRA”), which may be completed by the server 102 at the beginning of the plan year or obtained from the member at any other time. This HRA enables the system 100 to get a baseline on the member's needs and if appropriate, route them into a wellness program. The second is through the use of care reminders based on documented member needs as well as Healthcare Effectiveness Data and Information Set (HEDIS) gaps based on member demographics. To that end, the system 100 (via the server 102) may send reminders to members for upcoming prescription refills, upcoming physician appointments, as well as reminders to seek preventative care like colonoscopies and mammograms. More robust predictive interventions can come in to play by utilizing a combination of member demographics, claims information, prescription drug information, and lab results. With these data points in hand, members who meet criteria can be notified and directed back to their PCP for follow up before an acute issue occurs via the respective personal computing devices 122 of the members.

A third area of proactive outreach enabled by the system 100 relates to increased member satisfaction. For example, the system 100 enables improving “Star ratings” associated with Consumer Assessment of Healthcare Providers and Systems (CARPS) and other health outcome surveys (“HOSs”) by facilitating targeted interactions with members to improve overall member satisfaction with both a user of the system 100 (e.g., health insurance provider, health management platform provider, etc.) as well as the care that the members are receiving. For example, the system 100 may enable targeted surveys and interactions via the personal computing device 122 (e.g., by dynamically updating and personalizing the applications 112 and/or content 114 provided on each computing device 122), the user of the system 100 (via the server 102) monitors member satisfaction and emotional wellbeing, allowing interventions when appropriate.

The personal computing device 122 also enables members to interact with their insurance plan through tasks designed to improve wellness, which may ultimately align with a Centers for Medicare & Medicaid Services (CMS) stars rating program. In some examples, member interactions with the system 100 (via each member's computing device 122) may enable incentivizing members to take broader ownership of their healthcare and earn rewards for taking action on preventative wellness. Through the personal computing device 122, a user (e.g., health insurance provider, health management service provider, etc.) of the system 100 (via the server 102) will be able to interact with members to send reminders in a targeted manner, while also allow members to track their progress towards earning rewards.

Through a combination of HRA results, historic utilization and disease state information, the personal computing device 122 of each member enables operators of the system 100 (via the server 102) to customize the content at a member level to provide a personalized experience. This could include items like specific education tailored to members with diabetes, to mental wellbeing improvement for members with depression. This content customization enabled by the system 100 is a key differentiator against traditional static member information sharing systems.

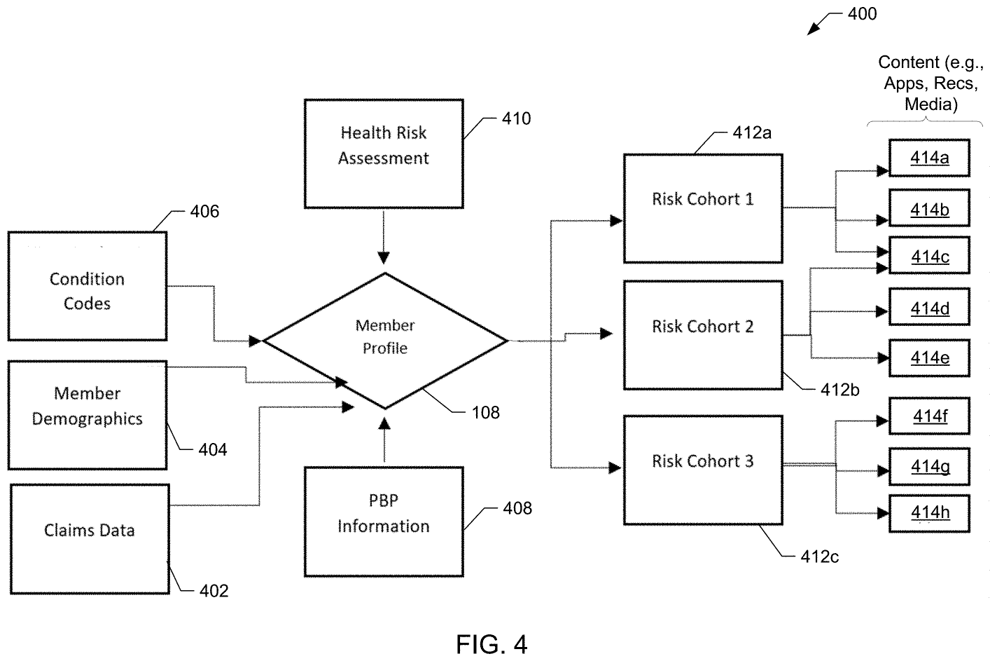

FIG. 4 shows a diagram of a process 400 performed by the health management server 102 of FIGS. 1-3 for creating a member profile 108, according to an example embodiment of the present disclosure. For example, the process 400 represented in FIG. 4 may represent an algorithm 400 (e.g., program instructions stored on a memory, etc.) executed by one or more processors of the server 102 to perform the functions of the process 400 described below.

The process 400, for each participating member, receives claims data 402, from, for example, the medical servers 104. The process 400 also receives member demographic data 404 from, for example, a member's registration information for an insurance provider (e.g., previously collected and stored in a data storage accessible to the server 102). The process 400 further receives medical condition codes 406 (e.g., ICD codes, etc.) and insurance plan information 408 (e.g., HMO, PPO, etc.). The process 300 may additionally receive risk assessment information, which may be related to an insurance underwriting analysis of a member or other health assessment/prediction based on at least some of the data 402, 404, 406, and/or 408.

As shown in FIG. 4, the process 400 uses the data 402, 404, 406, 408 and 410 to create a member profile 108. The process 400 then compares the member profile 108 to criteria for a plurality of risk cohort groups 412a, 412b, 412c (collectively referred to herein as risk cohort groups 412). The process 400 determines which of the risk cohort groups 412 matches the member profile 108. As an example, a first risk cohort group 412 may be associated with people who are smokers (e.g., as indicated by HRA answers, historical claims, etc.). As another example, a second risk cohort group 412 may be associated with people who are diagnosed with chronic obstructive pulmonary disease (COPD). As yet another example, a third risk cohort group 412 may be associated with people who are diagnosed with diabetes. Other examples are possible. In some examples, each of the risk cohort groups 412 corresponds to one or more disease conditions and/or combinations of disease conditions. Each risk cohort group 412 is assigned a different configuration of applications and/or other content (e.g., health recommendations, video content, audio content, etc.) for treating/managing the medical condition(s) associated with that group. In the illustrated example, each of the applications/content 414a, 412b, 412c, 414d, 414e, 414f, 414g, and 414h (collectively referred to herein as content 414) is assigned to one or more risk cohort groups 412.

It is noted that FIG. 4 shows three risk cohort groups 412 and three contents 414 assigned to each group 412 only for the sake of example. It should be appreciated that there may be fewer or more (e.g., ten, twenty, fifty, etc.) possible risk cohort groups 412, and/or fewer or more contents 414 assigned to each cohort group.

After assigning a member profile 108 to a risk cohort group 412, the example processes 400 identifies the applications and/or content that is assigned to the group. The process 400 also identifies a personal computing device (e.g., one of the devices 122 of FIG. 1) that is assigned to the member. The process 40 then installs the identified applications and/or other content onto the selected personal computing device 122. The process 400 may optionally also involve causing the personal computing device 122 to be provided to the member. This may include generating a mailing document and causing the personal computing device to be packaged and shipped. This may also include sending a voicemail, text message, and/or email message to the member regarding a location from which the personal computing device may be acquired. Alternatively or additionally, the process 400 may involve adding the identified content 414 to the personal computing device 122 of the member via network 105 (shown in FIG. 1).

Thus, it should be appreciated that the example process 400 updates a member profile 108 even after initial deployment. For example, as new data 402, 404, 406, 408, and/or 410 for a member is received, the process 400 updates the member profile 108. The process 400 may also check whether the updated member profile 108 should be assigned to the initially assigned (or previously assigned) risk cohort group 412. In some examples, if there is a change in assignment, the process 400 identifies the applications and/or content 414 associated with the newly assigned risk cohort group 412, and then performs an over-the-air (e.g., via the network 105) update such that applications and/or content associated with the newly assigned risk cohort group are installed. Further, the process 400 may uninstall applications and/or remove content that is not associated with the newly assigned risk cohort group 412. In some instances, some applications and/or content (e.g., 414c) may overlap between risk cohort groups such that these applications and/or content may already have been installed when a member changes between risk cohort groups 412. Accordingly, in some examples, the process 400 removes previously installed and/or loaded a particular content 414 from a computing device 122 based on the particular content 414 not being associated with a new risk cohort group 412 of the member associated with the computing device 122.

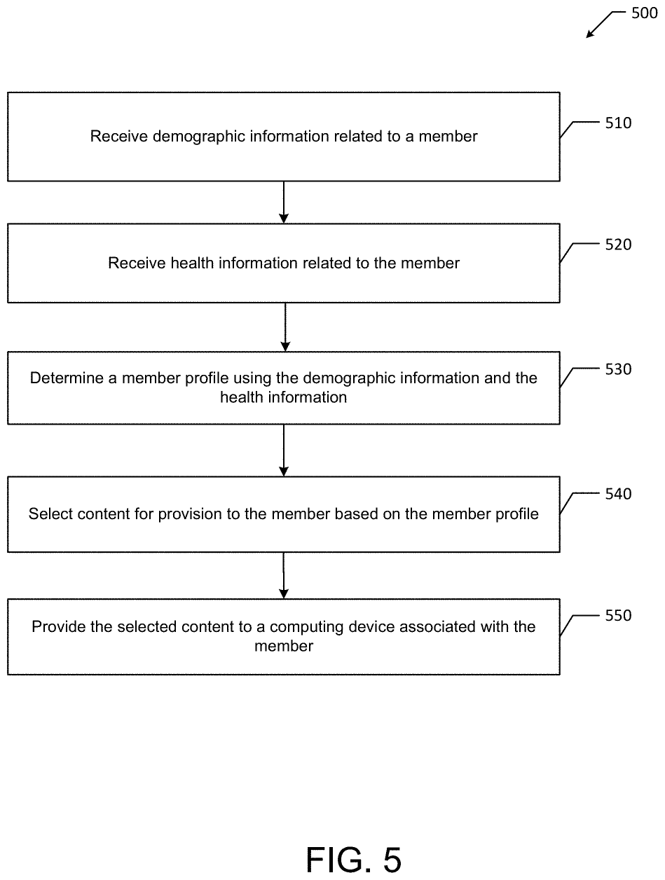

FIG. 5 illustrates a flowchart of an example method 500 for selectively providing content at a computing device associated with a member of a health management system, according to an example embodiment of the present disclosure. Although the example method 500 is described with reference to the flowchart illustrated in FIG. 5, it will be appreciated that many other methods of performing the acts associated with the method 600 may be used. For example, the order of some of the blocks may be changed, certain blocks may be combined with other blocks, one or more blocks may be repeated, and/or some of the blocks may be removed. The method 500 may be performed by processing logic that may comprise hardware (circuitry, dedicated logic, etc.), software, or a combination of both. For example, method 500 can be implemented using the health management server 102, and/or one or more other components illustrated in FIGS. 1-3.

At block 510, method 500 involves receiving demographic information related to a member. For example, the server 102 may receive the demographic information from one or more medical servers 104 and/or from data storage/memory 106. In some examples, the server 102 may obtain and/or determine at least some of the demographic information from health insurance plan documents previously submitted and/or generated for the member. A non-exhaustive list of example demographic information received at block 510 includes an actual age of the member (e.g., optionally computed based on a birth date of the member and a current date, etc.), a gender of the member, and/or an address of the member, among other examples. In some examples, the demographic information may also include a race, ethnicity, income level, or other internal (e.g., hidden) demographic information.

At block 520, method 500 involves receiving health information related to the member. For example, the server 102 may receive the health information from one or more medical servers 104 and/or from data storage/memory 106. In some examples, the server 102 may obtain and/or determine at least some of the health information from health insurance plan documents previously submitted and/or generated for the member. A non-exhaustive list of example health information received at block 520 includes one or more medical condition codes associated with medical conditions (e.g., chronic conditions, etc.) of the member, a health risk assessment (HRA), health insurance plan information (e.g., a health insurance plan identifier (ID), an effective date of the health insurance plan, etc.), and/or health insurance claim data, among other examples.

In an example, the health information includes medical condition codes (e.g., ICD codes) of medical conditions (e.g., COPD, heart disease, etc.) that the member currently has or previously had. This information can be obtained, for example, from insurance claims submitted by the member or by a medical provider, drug prescription records, and/or other data supplied by one or more medical providers (e.g., via medical servers 104) and/or by the member.

In another example, the health information includes an HRA of the member. For example, the member can be provided an HRA questionnaire that asks questions such as “How often do you feel depressed?” The member can then provide a response (e.g., “not at all,” several days every month,” etc.) that is mapped to a value (e.g., value between 0 to 100, etc.).

In another example, the health information includes health insurance plan information (e.g., PCP provider, number of annual checkups permitted, etc.).

In another example, the health information includes health insurance claim data (e.g., claim data submitted by a medical provider after a patient visit by the member, etc.).

At block 530, method 500 involves determining a member profile using the demographic information and the health information. For example, the server 102 can determine the member profile 108, in line with the discussion in the description of FIG. 4. The member profile may include external metrics (e.g., not hidden) such as the demographic information described above (e.g., age, gender, etc.) and/or internal metrics (e.g., hidden data) such as race, ethnicity, income level, etc.

In some examples, method 500 involves determining a health index score for the member based on the health information and the demographic information. In some instances, determining the member profile at block 530 may involve determining the health index score. As an example, the health index score for the member can be computed by the server 102 according to the following equation:

Health Index=HRA responses*Historical claims*Demographics*Socioeconomic factors.

In this equation, each HRA response may be assigned a value. For instance, an HRA questionnaire may ask a user “How often do you feel depressed?” In response, the user selects one of the following options: “Not at all,” “Several days a month,” “more days than not,” “nearly every day.” Each option is associated with a value that characterizes the response (e.g., 100, 60, 50, 40). Thus, depending on the responses selected by the member in the HRA questionnaire, corresponding values can be inserted as the “HRA responses” variable(s) of the equation above. Similarly, historical claims (e.g., diabetes medication, demographics (e.g., age, gender, etc.), and socioeconomic factors (e.g., address, ethnicity, income level, etc.) can be obtained from the member (e.g., via a questionnaire), from health insurance information, or from other sources (e.g., medical servers 104, etc.), and can also be assigned a corresponding value (e.g., between 0 and 100, etc.) in the equation above. As such, the health index score can be computed for each member as a single value that represents an overall health state of the member.

Accordingly, in some examples, method 500 involves determining the health index score as a weighted some of values, where the values characterize the heath information and/or the demographic information, in line with the discussion above.

It should be appreciated that the equation above is provided only for the sake of example. Thus, different computations of the health index score are possible without departing from the scope of the present disclosure. More generally, the health index can be any calculated number that represents a health state of the member based on the various demographic information and health information received at blocks 510 and 520.

In some examples, method 500 involves updating the health index in response to detecting an interaction by the member with the selected content. Referring back to FIG. 1 for example, the server 102 may increase the health index score of the member associated with device 122a if the member runs the application 112a or if the member accesses the content 114a (e.g., watches an educational video 114a, etc.). As another example, the server 102 may decrease the health index score if the member of device 122a does not run the application 112a and/or access the content 114a within a threshold period of time (e.g., a week, etc.).

In some examples method 500 involves determining a health age for the member based on the health information and the demographic information. The health age may be different from the actual age of the member. As an example, the health age of the member can be computed by the server 102 (e.g., at block 530) according to the following equation:

Health Age=Actual Age+(Average Death Age−Predicted Death Age)

In this equation, the Actual Age of the member (e.g., 70 years) can be obtained and/or computed using the demographic information received at block 510. The average death age may correspond to an average death age associated with the actual age of the member and/or a gender of the member. For example, if the member is a female that has a history of kidney failure and chronic kidney disease, the average death age in the equation above may be determined by server 102 based on an average death age of females (e.g., statistical average age of 76.2 years indicated by the center of disease control (CDC) for females, etc.). The predicted death age may correspond to an average death age of people that have similar medical histories as the member (e.g., predicted death age of 60 years based on statistics compiled for other people who had similar medical conditions as the member, etc.). Thus, in this example, the health age of the member may be computed by server 102 using the equation above as 70+(76.2−60)=86.2 years.

It should be appreciated that the equation above is provided only for the sake of example. Thus, different computations of the health age are possible without departing from the scope of the present disclosure. More generally, the health age can be any calculated number that represents a hypothetical age for the member that accounts for the member's health state. For example, the health age may be determined to be less than the actual age of the member if the health state of the member is determined to be better than a reference health state of an average person that has the same actual age. On the other hand, the health age may be determined to be more than the actual age of the member if the health state of the member is determined to be worse than the reference health state associated with the actual age.

Thus, in some examples, method 500 involves determining that a health state of the member is better than a reference health state associated with an actual age of the member, and determining the health age accordingly, in line with the discussion above.

At block 540, method 500 involves selecting content for provision to the member based on the member profile. Referring back to FIG. 1 for example, the server 102 may select content 114d (e.g., media, health recommendation, etc.) and/or application 112d for the computing device 112d associated with the member based on the specific health information and/or demographic information matching the selected content. For instance, referring back to FIG. 4, the selected content at block 540 may be mapped to a risk cohort group that matches the member profile 108, in line with the discussion in the description of FIG. 4.

In some examples, the selection at block 540 involves identifying an application from a plurality of applications indicated in a content database. Referring back to FIG. 1 for example, the server 102 may identify application 112d (e.g., smoking cessation software application, etc.) from the plurality of applications 112 indicated in the content database 110 based on the member profile (determined at block 530) indicating that the member is a smoker.

In some examples, method 500 involves selecting a risk cohort group for the member from a plurality of risk cohort groups based on the determined member profile. In these examples, selecting the content at block 540 may be further based on the content being associated with the determined risk cohort group. Referring back to FIG. 4 for example, the server 102 (shown in FIGS. 1-3) may select content 414a, 414b, and/or 414c based on the member profile 108 matching the risk cohort group 412a (e.g., where the member has a history of kidney disease and the group 412a assigns content 414a, 414b, 414c for members that have kidney diseases, etc.).

In some examples, the selection at block 540 involves selecting media content (e.g., educational videos, educational audio recordings, educational reading material, etc.) from a content database (e.g., database 110) based on the member profile. For example, the server 102 may select a link to a fitness or exercise video (i.e., the selected content) from the content database 110 appropriate for improving or maintaining the member's health state.

At block 550, method 500 involves providing the selected content to a computing device associated with the member. Referring back to FIG. 1 for example, the server 102 may provide the selected content (e.g., application installation instructions, medical visit reminders, telehealth call reminders, etc.) to a computing device 122 of the member (e.g., via the network 105). As another example, referring back to FIGS. 2-3, the server 102 may pre-install the selected content (e.g., application 112d, content 114d, etc.) onto the computing device 122d of the member before initial deployment (e.g., provision, mailing, etc.) of the computing device 122d to the member.

In some examples, where method 500 involves determining a health index score in line with the discussion above, method 500 may also involve providing the health index score for display at the computing device. For example, the server 102 can instruct a graphical user interface (e.g., an application, website, member portal, etc.) displayed on a computing device 122 of the member to display the computed health index score.

In some examples, where method 500 involves identifying an application for the member, providing the selected content at block 550 also involves installing the identified application on the computing device of the member. As an example, referring back to FIGS. 2-3, the server 102 may pre-install the identified application 112a onto computing device 122a while the computing device 122a is in the repository 120 prior to initial deployment (e.g., provision, mailing, etc.) of the computing device 122a to the member. As another example, referring back to FIG. 1, the server 102, may provide (via the network 105) instructions to the computing device 122d that cause an operating system of the computing device 122d to download (e.g., from a content server 116), install, and/or load the application 112d onto the computing device 122d.

In some examples, where method 500 involves generating a health recommendation, method 500 also involves providing the health recommendation for display at the computing device of the member. Referring back to FIG. 1 for example, the server 102 may instruct computing device 122b to display a summary of the member profile 108b of the member that includes the health recommendation (e.g., reminder to schedule a PCP visit via a telehealth service or with a clinical team, healthy eating or fitness suggestions, reminders about fitness benefits available to the member in the members health insurance plan, etc.).

In some examples, where method 500 involves determining a health age for the member, providing the selected content at block 550 also involves providing the health age for display at the computing device of the member. For example, referring back to FIG. 1, the server 102 may instruct computing device 122b to display a summary of the member profile 108b of the member that includes the health age (e.g., computed by the server 102 in line with the description at block 530).

In some examples, method 500 involves onboarding a member with a newly received personal computing device 122. Referring back to FIGS. 1-2 for example, the server 102 may determine that a member has requested a computing device 122 (from the computing devices in the repository 120). The health management server 102 then pre-loads the personal computing device 122 with the identified applications 112 and/or content 114 selected for the member at block 540. The computing device 122 is then mailed or otherwise provided to the member. Upon powering the computing device 122, a welcome video (e.g., content 114 pre-installed by the server 102 on the computing device 122) may be displayed and/or an interactive session is initiated to help familiarize the member with the applications 112 and/or content 114 on their personal computing device 122.

More generally, the example personal computing device 122 can be loaded (prior to deployment or at a later time) by the server 102 with applications 112 and/or content 114 that allow the member to be proactive in managing their health care needs. This includes applications for on-demand primary care services, for instance. Through an application 112 selected and/or installed on the computing device 122 of the member, the member may be able to initiate a visit with a primary care physician 24 hours a day, 7 days a week, for instance. The visits start out as a text based interaction (on the computing device 122 of the member) and can transition to an audio or video visit via the computing device 122 if necessary. This telehealth offering combined with improved access via the personal computing device 122 of the member can help prevent unnecessary trips to an emergency room and ultimately allows members to seek care in a more cost effective way, for instance. The applications 112 also enable members to interact with other supplemental benefit offerings. This includes scheduling rides for doctor appointments and trips to a pharmacy or conducting a digital hearing exam via the personal computing device 122 for ordering a hearing aid. By providing members with this tool, the personal computing device 122 enables members greater access to care that ultimately will drive higher member satisfaction and improved health outcomes.

In some examples, method 500 involves detecting a clinical event and responsively updating the member profile, health index score, and/or health age of the member. Certain clinical events may trigger outreach (e.g., by the server 102) to a member via a respective personal computing device 122 and enable a Health Services team to engage with the member based on the triggering event. For example, if the server 102 detects an emergency room visit and/or hospital discharge (e.g., reported by a medical server 104, reported in health insurance claim data, etc.), the server 102 may redirect members back to their Primary Care Physician (“PCP”) for a checkup, etc. Interventions in the time period immediately following these events are sometimes critical to reducing avoidable Emergency Room (ER) visits and readmissions as their PCP is able to perform medical reconciliation as well as verify that the acute issue that led to the initial visit or admission has been resolved. Thus, example systems, methods, and apparatus herein also enable the PCP to implement a care plan proactively with the member to help prevent acute issues from arising in the future.

In some examples, method 500 also involves the server 102 reviewing prescription drug fills associated with the member and monitoring for potentially dangerous interactions between different prescription drugs. For example, the server 102 can use the personal computing device 122 of the member to conduct outreach to members for purposes of lead disease management, redirection to the member's PCP for resolution, or other outreach actions by a case management health team via the server 102 and the computing device 122 of the member.

In some examples, method 600 also involves providing, for display at the computing device of the member, an aggregated view of at least part of the member profile, the demographic information, and the health information. For example, the server 102 may provide instructions and information to the computing device 122 of the member to cause the computing device 122 to display a user interface (e.g., web portal, website, application graphical user interface, etc.) at the computing device 122 of the member.

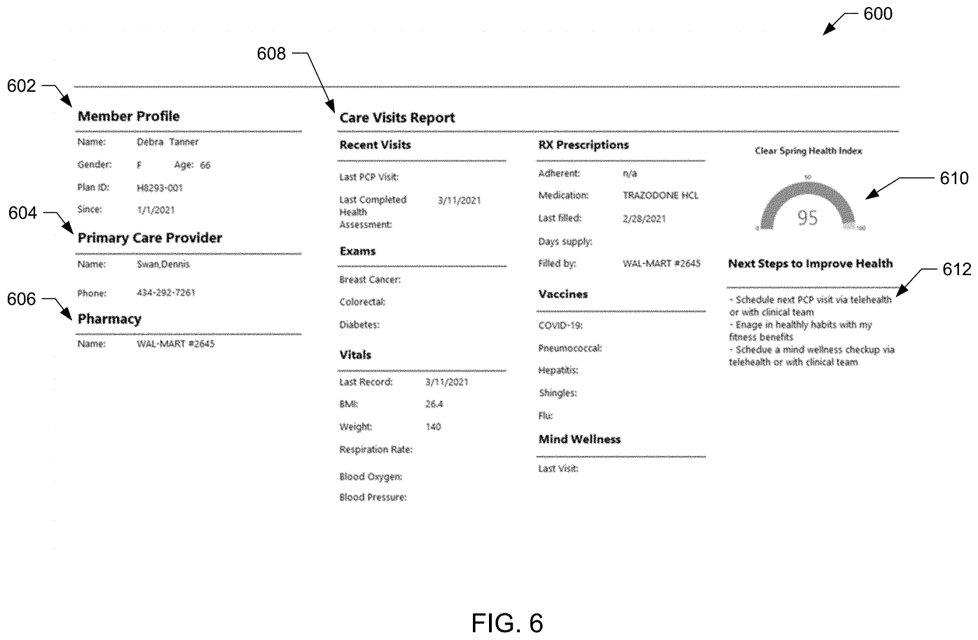

FIG. 6 illustrates an example user interface 600 provided at a computing device 122 of a member, according to an example embodiment of the present disclosure. For example, referring back to FIG. 1, the user interface 600 may correspond to a graphical user interface provided by the system 100 and displayed at a computing device 122b of a member based on information in the member profile 108b of the member (generated by the server 102 and provided to the device 122b via the network 105).

As shown, the user interface (UI) 600 includes a member profile summary 602, which displays demographic information (such as the demographic information received at block 510 of the method 500). The UI 600 also includes primary care provider information 604, pharmacy information 606, and health information 608. The UI 600 also includes a health index score 610 that is computed (and optionally dynamically updated) for the member, in line with the discussion in the description of block 530 of method 500. The UI 600 also includes a plurality of health recommendations 612, which may correspond to the selected content described at blocks 540-550 of method 500. In the illustrated example, health recommendations 612 include three recommendations (e.g., schedule next PCP visit, engage in healthy habits, schedule a mind wellness checkup, etc.) that may be generated and/or selected by the server 102 (e.g., at blocks 530-550 of method 500) based on the health information and the demographic information (received at blocks 510-520 of method 500). However, it should be appreciated that the UI 600 may include fewer, more, or different health recommendations 612 without departing from the scope of the present disclosure.

In some examples, the information 602-612 displayed in the UI 600 can be compiled (and/or monitored) by the server 102 from multiple sources (e.g., medical servers 104, health insurance plan information, HRA information, health insurance claim data, etc.), and aggregated into a combined representation as illustrated in the UI 600. In some examples, the system 100 and/or the server 102 dynamically updates any of the information 602-612 displayed in the UI 600, for example, in response to receiving updated demographic and/or health information (e.g., clinical visit events, etc.) for the member, such as updates to any of the demographic and health information described at blocks 510 and 520 of method 500.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention. The terms “comprise”, “comprises”, “comprised” or “comprising”, “including” or “having” and the like in the present specification and claims are used in an inclusive sense, that is to specify the presence of the stated features but not preclude the presence of additional or further features. It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

What is claimed is:

1. A method for selective content provision for members of a health management system, the method comprising:
receiving demographic information related to a member;
receiving health information related to the member, the health information including a medical condition code, a health risk assessment, health insurance plan information, and health insurance claim data;
determining a member profile using the demographic information and the health information;
selecting content for the member based on the member profile; and
providing the selected content to a computing device associated with the member.

2. The method of claim 1, further comprising:
determining a health index score for the member based on the health information and the demographic information, wherein determining the member profile includes determining the health index score.

3. The method of claim 2, wherein determining the health index score includes determining a weighted sum of values, the values characterizing the health information and the demographic information.

4. The method of claim 2, wherein providing the selected content at the computing device includes providing the health index score for display at the computing device.

5. The method of claim 2, further comprising:
updating the health index in response to detecting an interaction by the member with the selected content, wherein determining the member profile includes updating the member profile based on the updated health index.

6. The method of claim 5, further comprising:
increasing the health index in response to a determination that the member successfully completed a task associated with the selected content provided at the computing device.

7. The method of claim 5, further comprising:
decreasing the health index in response to a determination of failure to complete a task associated with the selected content provided at the computing device.

8. The method of claim 1, further comprising:
identifying an application from a plurality of applications indicated in a content database, wherein selecting the content includes identifying the application; and
installing the identified application on the computing device of the member, wherein providing the selected content includes installing the identified application.

9. The method of claim 1, further comprising:
selecting a risk cohort group for the member from a plurality of risk cohort groups based on the determined member profile, wherein selecting the content is further based on the content being associated with the determined risk cohort group.

10. The method of claim 1, wherein the health risk assessment includes at least one of underwriting data, health condition results from predictive modeling, or morbidity assessment information.

11. The method of claim 1, further comprising causing the computing device to be provided to the member.

12. The method of claim 1, wherein selecting the content includes selecting media content from a content database based on the member profile, and wherein providing the selected content includes providing the selected media content to the computing device of the member via a network.

13. The method of claim 1, further comprising:
generating a health recommendation for the member based on the member profile; and
providing the health recommendation for display at the computing device of the member, wherein providing the selected content includes providing the health recommendation.

14. The method of claim 1, further comprising:
determining a health age for the member based on the health information and the demographic information, wherein the determined health age is different from an actual age of the member; and
providing the health age for display at the computing device, wherein providing the selected content comprises providing the health age.

15. The method of claim 14, further comprising:
determining, based on the health information and the demographic information, that a health state of the member is better than a reference health state associated with an actual age of the member,
wherein the health age is determined to be less than the actual age based on the determination that the health state of the member is better than the reference health state.

16. A system comprising:
a content database; and
a health management server configured to:
receive demographic information related to a member;
receive health information related to the member, the health information including a medical condition code, a health risk assessment, health insurance plan information, and health insurance claim data;
determine a member profile using the demographic information and the health information;
select, from the content database, content for the member based on the member profile; and
load the selected content onto a computing device designated for the member.

17. The system of claim 16, wherein the health management server is configured to receive the health information from one or more medical servers via a network.

18. The system of claim 16, further comprising:
a member profile database, wherein the health management server is configured to store the member profile of the member in the member profile database.

19. A non-transitory computer readable medium storing instructions that, when executed by one or more processors, cause a computing system to:
receive demographic information related to a member;
receive health information related to the member, the health information including a medical condition code, a health risk assessment, health insurance plan information, and health insurance claim data;
determine a member profile using the demographic information and the health information;
select content for the member based on the member profile; and
provide, via a network, the selected content to a computing device associated with the member.

20. The non-transitory computer readable medium of claim 19, wherein execution of the instructions further causes the computing system to:
determine a health index score for the member based on the health information and the demographic information, wherein determining the member profile includes determining the health index score.
